(19) United States
(12) Patent Application Publication
(10) Pub. No.: US 2015/0080361 A1
(43) Pub. Date: Mar. 19, 2015

(54) VETERINARY COMPOSITIONS

(71) Applicant: Zoetis LLC, Florham Park, NJ (US)

(72) Inventors: Sunil Thomas Kumar Narishetty, Kalamazoo, MI (US); Jeffrey Ellis Price, Kalamazoo, MI (US); Sreenath Repakula, Mississauga (CA)

(21) Appl. No.: 14/452,862

(22) Filed: Aug. 6, 2014

Related U.S. Application Data
(63) Continuation of application No. 13/580,156, filed on Aug. 21, 2012, now abandoned.
(60) Provisional application No. 61/307,713, filed on Feb. 24, 2010.

(51) Int. Cl. A61K 31/519; A61K 9/00; A61K 9/20; A61K 31/197; A61K 31/43

(52) U.S. Cl. CPC A61K 31/519; A61K 31/197; A61K 31/43; A61K 9/2054; A61K 9/2031; A61K 9/2027; A61K 9/2059; A61K 9/0065
USPC 514/197; 514/265.1; 514/561

(57) ABSTRACT

The present invention relates to veterinary compositions in a form of an orally deliverable tablet, and more particularly to a controlled-release composition that provides sufficiently long duration to permit once daily administration.

### CROSS-REFERENCE TO RELATED APPLICATIONS

This application is a continuation application of U.S. patent application Ser. No. 13/580,156, filed Aug. 21, 2012, now pending, which is the National Stage of International Application No. PCT/IB2011/050625, filed Feb. 15, 2011, now abandoned, which claims priority to U.S. Provisional Application No. 61/307,713, filed Feb. 24, 2010, now abandoned.

### FIELD OF THE INVENTION

The present invention relates to veterinary compositions in a form of an orally deliverable tablet, and more particularly to a controlled-release composition that provides sufficiently long duration to permit once daily administration.

### BACKGROUND OF THE INVENTION

Extended time release technology for drug molecules has been extensively studied and developed since early 1950s. Oral controlled release dosage forms have been used to improve therapy of many important human medications with commercial successes.

However, traditional controlled release dosage forms developed for humans do not function as intended when used similarly in canines. Canines have stronger muscular forces in the stomach when compared to humans. Additionally, canines have much shorter gastrointestinal (GI) tracts (about half the length as humans); therefore, shorter GI tract transit time. The combination of higher forces and shorter GI tract transit time in canines make the conventional controlled release tablets designed for humans unsuitable for dogs. Further, canine's stomach has the pylorus, the region of the stomach that connects to the first section of the small intestine in mammals, at the top of the stomach wherein humans have the pylorus at the bottom of the stomach as illustrated in FIG. 1. Therefore, canines' physiological differences require a novel non-buoyant approach to gastric retention. A controlled release dosage tablet must sink to the bottom of the stomach and should not have buoyant or floating properties. A tablet's “sinking behavior” upon swallowing followed by rapid hydration is necessary to keep the tablet away from the pylorus opening thereby preventing it from easily slipping through the stomach.

To date, there are no solid oral controlled release tablet dosage forms on the market for dosing canines on a once daily schedule. As a result, there are unresolved needs to develop a novel controlled-release composition in a form of an orally deliverable tablet that can be retained in the canine stomach for a prolonged time for absorption and survive the increased muscular forces experienced in a canine's stomach. The present invention provides veterinary compositions in a form of orally deliverable tablets with prolonged gastric retention that is suitable for once-daily oral administration in canines.

### SUMMARY OF THE INVENTION

The present invention is directed to a controlled-release veterinary composition in a form of an orally deliverable tablet. The tablet of the present invention uses high molecular weight or high viscosity polymers that are sufficient to withstand the GI forces of a canine's stomach. Upon swallowing, the tablets of the present invention sink to the bottom of the canine stomach and rapidly hydrate to provide prolonged gastric retention that is suitable for once-daily oral administration in canines.

Specifically, the veterinary composition of the present invention comprises:

(a) at least one bioactive agent for veterinary use;
(b) a polymer having a viscosity in a range of from about 80,000 to about 120,000 mPa·s, or a polymer having molecular weight from about 1,000,000 to about 9,000,000 daltons, in an amount of about 5% to about 60% of the total weight of the tablet; and
(c) at least one disintegrant agent in an amount between about 10% to about 50% of the total weight of the tablet.

The present invention further provides methods for preparing a controlled-release veterinary composition in a form of an orally deliverable tablet.

The present invention further provides methods for treating canines having a condition or disorder for which at least one bioactive agent is needed; the methods comprise orally administering to canines a veterinary composition in a form of orally deliverable tablet.

### BRIEF DESCRIPTION OF THE DRAWINGS

FIG. 1 Illustrates that canine's stomach has the pylorus at the top of the stomach wherein humans have the pylorus at the bottom of the stomach.

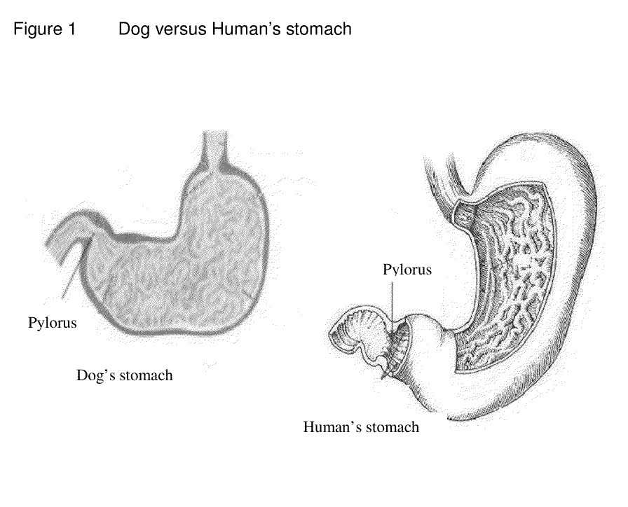

FIG. 2 illustrates in vitro dissolution profiles of Examples 1 and 2.

FIG. 3 illustrates in vitro dissolution profiles of pregabalin tablets of the present invention and the immediate release tablet.

FIG. 4 illustrates in vitro dissolution profiles of amoxicillin trihydrate tablet of the present.

FIG. 5 illustrates in vitro dissolution profiles of tramadol tablet of the present invention and the immediate release tablet.

FIG. 6 illustrates mean plasma concentrations of Compound A in dogs v. time for Example 1.

FIG. 7 illustrates mean plasma concentrations of Compound A in dogs v. time for Example 2.

FIG. 8 illustrates mean plasma concentrations of pregabalin in dogs v. time.

FIG. 9 mean plasma concentrations of amoxicillin in dog v. time.

FIG. 10 illustrates a non-buoyant “sinking tablet” of Example 2 in a beaker of citrate buffer.

### DETAILED DESCRIPTION OF THE INVENTION

### Bioactive Agents

Suitable bioactive agents of the present invention are a compound, or its acceptable salt or prodrug that has sufficient aqueous solubility. Typically, the bioactive agents suitable herein need solubility more than 0.1 mg/mL or above. The term “solubility” herein means solubility in water at 20-25° C. at any physiologically acceptable pH, for example at any pH in the range of about 3 to about 8.

The bioactive agents of the present invention can be of any therapeutic category for veterinary use, for example, any of the therapeutic categories listed in The Merck Index, 16^{th} edition (2006), provides that the bioactive agents possess sufficient aqueous solubility more than 0.1 mg/mL or above. Bioactive agents useful herein can be in the therapeutic category including, but not limited to, analgesics, anti-inflammatory agents, anti-parasitic, anthelmintics, endectocides, antiemetic, anti-microbials, anti-fungal and anti-viral agents, antihistamines, anti-allergic agents, pain relievers, sedatives and tranquilizers, respiratory stimulants, muscle relaxants, weight control and loss agents, anti-diabetic, vitamins and mineral supplements, hormones, immunostimulants and immunosuppressants, sleeping aids, dermatologic including anti-pruitic, behavior modification drugs, anticonvulsant, and combinations thereof.

A partial list of bioactive agents for illustration includes, but not limited to, maropitant citrate, preferably under the trade name Cerenia™ Tablets, amoxicillin, preferably under the trade name Amoxi-TABS®, dexmedetomidine hydrochloride, preferably under the trade name Dexdomitor®, tulathromycin, preferably under the trade name Draxxin®, selamectin, preferably under the trade name Revolution®, ceftiofur, lincomycin hydrochloride, tramadol, pregabalin, Janus Kinase (JAK) inhibitors, aspirin, ibuprofen, morphine, spectinomycin, buprenorphine, furosemide, ketoprofen, marbofloxacin, selegiline HCl & L-deprenyl HCl, cefpodoxime proxetil, trimeprazine tartrate, prednisolone, clinafloxacin, epsiprantel, amoxicillin trihydrate/clavulanate potassium, diclofenac sodium, primidone, deracoxib, diphendydramine, methocarbamol, chloramphenicol, tetracycline, penicillin VK, phenylbutazone, butorphanol tartrate, cefadroxil, oxycodone, clindamycin, doxylamine succinate, aminopropazine fumarate & neomycin sulfate, isopropamide iodide, liothyronine sodium, thenium closylate, methenamine mandelate & sulfamethizole, sulfachlorpyridazine, chlorphenesin carbamate, or combination thereof. Bioactive agents that are suitable of combination use for the present invention include maropitant citrate.

All bioactive agents useful herein can be prepared by methods known to those skilled in the art, including methods disclosed in patents, published patent applications and other literature pertaining to specific bioactive agents of interest.

Specific agents useful herein further include Janus Kinase (JAK) inhibitors of formula I:

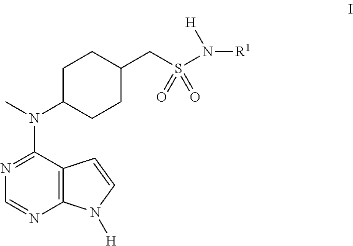

or an acceptable salt thereof wherein R¹ is —C₁₋₄alkyl, optionally substituted with hydroxy. Specifically, the agent useful herein is N-methyl-1-{trans-4-[methyl(7H-pyrrolo[2,3-d]pyrimidin-4-yl)amino]cyclohexyl}methanesulfonamide (hereinafter as Compound A), or its acceptable salts thereof. Methods of preparing a JAK compound of formula I, or its acceptable salt thereof are described in U.S. patent application Ser. No. 12/542,451, Pub. No. US2010/0075996, incorporated herein by reference. The veterinary composition of the present invention containing the JAK compounds of formula I or its pharmaceutically acceptable salts can be used to treat a variety of conditions or disease including allergic reactions, allergic dermatitis, atopic dermatitis, eczema, pruritus and other pruritic conditions and inflammatory diseases in animal including canine. One of the objects of the present invention is to use the veterinary composition of the present invention containing a compound of formula I for manufacturing of a medicament for the treatment of a variety of conditions or diseases such as allergic reactions, allergic dermatitis, atopic dermatitis, eczema, pruritus and other pruritic conditions and inflammatory diseases in animals including canine. Another object of the present invention is to provide a method for the treatment of a variety of conditions or diseases such as allergic reactions, allergic dermatitis, atopic dermatitis, eczema, pruritus and other pruritic conditions and inflammatory diseases in animals including dogs by administering to the animals in need an effective amount of the veterinary composition if the present invention containing a compound of formula I.

The amount of the bioactive agents for the veterinary composition in a form of oral tablets may be varied depending upon the potency of the particular compound, the solubility of an agent and the desired concentration, but is sufficient to provide a daily dose in one tablet. Determination of a therapeutically effective amount is well within the capability of those skilled in the art. Generally, the amount of therapeutic agents will range from 0.1% to 60% by weight of the tablet as a whole. Preferably, the amount of therapeutic agents will range from about 1% to about 40%, more preferably about 1% to about 25%, even more preferably from about 2% to 10% by weight of the tablet as a whole.

### Tablets

Orally deliverable tablets of the present invention can be of any suitable size and shape, for example round, oval, polygonal or pillow-shaped, and optionally bear nonfunctional surface markings.

The term “orally deliverable” herein means suitable for oral, including peroral and intra-oral (e.g., sublingual or buccal) administration, but tablets of the present invention are adapted primarily for oral administration, i. e., for swallowing, typically whole or broken, with the aid of food, water or other drinkable fluid.

Approximate sizes of the tablets described herein may be adjusted depending upon the weight of a dog in need. Generally, approximate tablet size is in a range from about 100 mg to about 1.5 g, preferably from about 250 mg to about 1 g, for a dog weight about 10 kg (about 20 pounds); from about 400 mg to about 3 g, preferably from about 750 mg to about 2 g, for a dog weight about 20 kg (about 40 pounds); from about 600 mg to about 5 g, preferably from about 1 g to about 3.5 g for a dog weight about 40 kg (about 80 pounds); and from about 1.5 g to about 7 g, preferably from about 2 g to about 5 g for a dog weight about 80 kg (about 160 pounds).

### Compositions

Polymers useful herein can be any of the materials in dosage forms as matrix-forming agents that have high molecular weight. The term “viscosity” is used to measure the rate at which a polymer solution flows—the slower a solution moves, the thicker it is—and the polymer molecular weight influences the viscosity. Viscosity of a polymer solution depends on the solvent and the temperature; in this case it refers to a 2% of the polymer aqueous solution. Polymers with high molecular weight or high viscosity are sufficient to withstand the GI forces of a canine's stomach and to modulate the release of a bioactive agent(s). Polymers useful herein typically have a molecular weight from about 1,000,000 to about 9,000,000 daltons, preferably from about 2,000,000 to about 4,000,000 daltons; or typically have an apparent viscosity from about 80,000 to about 120,000 milli Pascal Second (mPa·s). Human dosage forms typically use lower molecular weight or low viscosity polymers because they do not experience the increased gastric forces as found in a canine's stomach. Therefore, controlled release can be readily achieved in humans without using higher molecular weight or higher viscosity polymers. Further, lower molecular weight or lower viscosity controlled release polymers used in human dosage forms hydrate more readily without the need for disintegrants and have sufficient time to release drug while in the GI tract (due to its overall length) providing sufficient resonance time for once daily dosing.

Examples of such polymers of the present invention include, but are not limited to, methyl cellulose, carboxymethyl-cellulose sodium, crosslinked carboxymethylcellulose sodium, crosslinked hydroxypropylcellulose, hydroxypropylmethylcellulose, carboxymethyl starch, polymethacrylate, polyvinylpyrrolidone, polyvinyl alcohols, polyethylene glycols, potassium methacrylate-divinyl benzene copolymer, carboxymethylcellulose, alginates, albumin, gelatine, crosslinked polyvinylpyrrolidone, polyesters, polyanhydrides, scleroglucan, polymethylvinylether/anhydride copolymers, glucan, mannan, betacyclodextrins and cyclodextrin derivatives containing linear and/or branched polymeric chains and mixtures thereof. All of them are commercially available.

In one embodiment, the polymer useful herein is hydroxypropyl methyl cellulose (HPMC) having a viscosity of 80,000 or above, preferably known as Hypromellose 2208, or substantially equivalent products. In another embodiment, the polymer useful herein is high molecular weight polyethylene oxides (PEO), preferably known as Polyox WSR n-60k, or substantially equivalent products. Hypromellose 2208 and Polyox WSR n-60k, are commercially available polymers.

Generally, the quantity of a polymer of the composition of the present invention is in an amount from about 5 to about 80%, preferably from about 15% to about 50%, more preferably from about 20% to about 40%, by weight of the tablet as a whole. In a case of Hypromellose 2208, the preferred amount is in the range from about 25% to about 40% by weight of the tablet as a whole. In a case of Polyox WSR n-60k, the preferred amount is in the range from about 15% to about 35% by weight of the tablet as a whole.

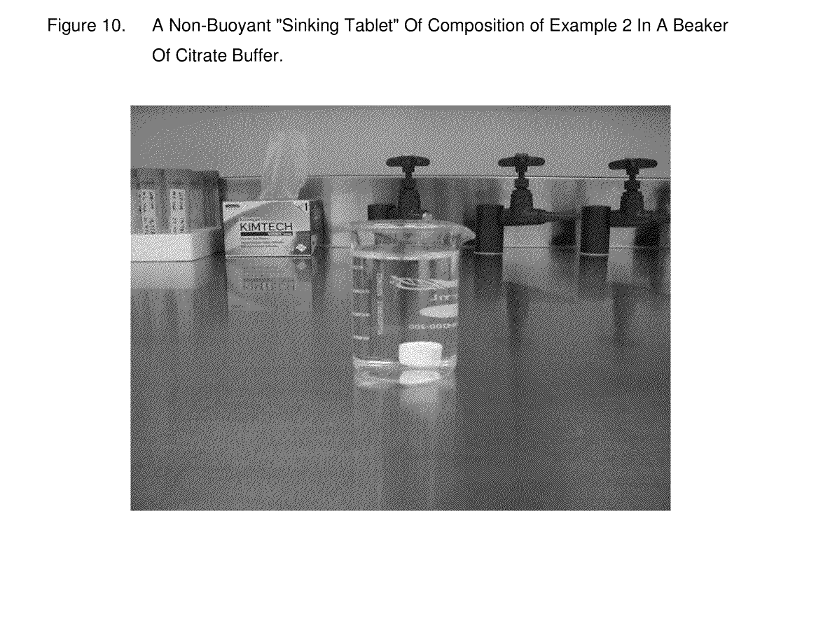

The term “disintegrants” useful herein refers to substances that rapidly swell, hydrate, and change volume or form upon contact with water within a short period of time, typically within 60 seconds or less. At least one high amount of disintegrant is present to the orally deliverable tablet of the present invention. The disintegrant provides very rapid swelling of the high molecular weight polymers. The tablets are easily swallowed and reach a significantly larger size in the stomach due to hydration and rapid swelling upon dosing. This inhibits the passage of the tablet through the pylorus; as a result the tablet is retained in the canine stomach facilitating a controlled release. Additionally, tablet's “sinking behavior” is now possible. FIG. 10 illustrates a non-buoyant “sinking tablet” of composition of Example 2 in a beaker of citrate buffer. After rapid tablet swelling, gelling is typically observed as a result of using a disintegrant at high levels. This increases the density of the hydrated and gelled tablets. The tablets sink to the bottom of the stomach, which prolong their gastric retention time. In conventional immediate release dosage forms the “gelling phenomenon” is not desired as it is known to cause drug release problems. It is believed, without being bound by theory, that the “gelling phenomenon” provides added benefit to the gastro retentiveness (through gelling) and improves drug release in controlled release dosage forms formulated with high molecular weight polymers. It is an unexpected result. In one embodiment, the disintegrant useful herein is croscarmellose sodium. In another embodiment, the disintegrant useful herein is sodium carboxymethyl starch. In another embodiment, the disintegrant useful herein is cross linked povidone. In another embodiment, the disintegrant useful herein is 2-hydroxypropyl ether (low substituted). Generally, the quantity of a disintegrant of the present invention is in an amount from about 10% to about 50%, preferably from about 10% to about 40%, more preferably from about 10% to about 25%, by weight of the tablet as a whole.

The composition of the present invention may further comprise veterinary acceptable excipients such as binders, fillers, diluents, water, pH buffering agents, glidants, adhesives or antiadherents, film coating materials, ionic or enteric polymers, non-ionic polymers, cellulose polymers, calcium salts, copolymers, sugars, alcohols, lubricants, colorants, stabilizers, surfactants, flavorants, preservatives, anti-oxidants, and combinations thereof.

Examples of binders include, but are not limited to, microcrystalline cellulose, pregelatinized starch, and polyvinyl pyrollidone.

Examples of diluents include, but are not limited to, microcrystalline cellulose, lactose, dicalcium phosphate, mannitol and water.

Examples of gelling agents include, but are not limited to, carbomer and polyethylene glycols.

Examples of enteric fillers or enteric polymers include, but are not limited to, methacrylate copolymers, cellulose acetate phthalate, and hydroxypropyl methyl cellulose acetate phthalate. Preferably, the enteric fillers or polymers have a pH range of about 5.5-9.0, more preferably about pH 5.5.

Examples of pH buffering agents include, but are not limited to, citric acid, sodium citrate, and disodium phosphate.

Examples of lubricants include, but are not limited to, magnesium stearate, sodium stearyl fumarate, and stearic acid.

### Method of Preparations

The veterinary composition in the form of an orally deliverable tablet described herein can be prepared using techniques well known in the art such as mixing a bioactive agent with a suitable polymer, a suitable disintegrant agent and other excipients. The mixture is subsequently blended or granulated and compressed to form a tablet. In one embodiment, a method for preparation of the present invention comprises the steps of: 1) weighing and placing all ingredients into suitable containers, 2) adding a suitable diluent to a mortar & pestle, 3) blending for 15 seconds to coat the mortar, 4) adding a bioactive, further blending, and then passing the mixture through a mesh screen, 5) lubricating the blend, and 6) compressing the lubricated powder blend into tablets using a suitable tablet press.

### EXAMPLES

The present invention will be further understood by reference to the following non-limiting examples 1-7 in the form of solid tablets prepared by direct compression.

### Example 1

| Ingredients | mg/tab | % | Function | Range % |
|---|---|---|---|---|
| Compound A | 10.75* | 2.15 | bioactive agent | 2-40 |
| maleate salt | | | | |
| Microcrystalline | 34.25 | 6.85 | binder/filler | 1-80 |
| cellulose | | | | |
| Hypromellose | 150.00 | 30.00 | control release | 5-60 |
| 2208 | | | polymer | |
| croscarmellose | 50.00 | 10.00 | disintegrant | 10-50 |
| sodium | | | | |
| polymethacrylate | 250.00 | 50.00 | enteric filler pH 5.5 | 1-75 |
| L100-55 | | | | |
| magnesium stearate | 5.00 | 1.00 | lubricant | 0.25-2 |
| Total Tablet Weight | 500.00 | 100.00 | | |

*10.75 mg of compound A maleate salt is 8 mg of free base equivalent.

### Example 2

| Ingredients | mg/tab | % | Function | Range % |
|---|---|---|---|---|
| Compound A maleate salt | 10.75* | 2.15 | bioactive agent | 2-40 |
| 8 parts polyvinyl acetate & 2 | 236.25 | 47.25 | binder/filler/rigidity | 1-80 |
| parts polyvinylpyrrolidone or | | | enhancer | |
| Kollidon SR | | | | |
| Crospovidone | 125.00 | 25.00 | disintegrant | 10-50 |
| Polyethylene oxide WSR N-60K | 100.00 | 20.00 | control release polymer | 5-60 |
| NF | | | | |
| Carbomer 71G or 971P | 25.00 | 5.00 | gelling | 0.5-20 |
| | | | agent/mucoadhesive | |
| magnesium stearate NF | 3.00 | 0.60 | lubricant | 0.25-2 |
| Total Tablet Weight | 500.00 | 100.00 | | |

*10.75 mg of compound A maleate salt is 8 mg of free base equivalent.

### Example 3

| Ingredients | mg/tab | % | Function | Range % |
|---|---|---|---|---|
| Pregabalin | 45.1* | 9.02 | bioactive agent | 2-40 |
| Microcrystalline | 49.9 | 9.98 | binder/filler | 1-80 |
| cellulose | | | | |
| Hypromellose 2208 | 200 | 40 | control release | 5-60 |
| | | | polymer | |
| croscarmellose | 50.00 | 10.00 | disintegrant | 10-50 |
| sodium | | | | |
| polymethacrylate | 150 | 30.00 | enteric filler pH 5.5 | 1-75 |
| L100-55 | | | | |
| magnesium | 5.00 | 1.00 | lubricant | 0.25-2 |
| stearate | | | | |
| Total Tablet | 500.00 | 100.00 | | |
| Weight | | | | |

*45.1 mg of Pregabalin is based on purity equivalent to 45 mg.

### Example 4

| Ingredients | mg/tab | % | Function | Range % |
|---|---|---|---|---|
| Pregabalin | 45.1* | 9.02 | bioactive agent | 2-40 |
| 8 parts polyvinyl acetate & 2 | 139.4 | 27.88 | binder/filler/rigidity | 1-80 |
| parts polyvinylpyrrolidone or | | | enhancer | |
| Kollidon SR | | | | |
| Crospovidone | 125.00 | 25.00 | disintegrant | 10-50 |
| Polyethylene oxide WSR | 150.00 | 30.00 | control release | 5-60 |
| N-60K NF | | | polymer/ | |
| Carbomer 71G or 971P | 37.5 | 7.5 | gelling agent/ | 0.5-20 |
| | | | mucoadhesive | |
| magnesium stearate NF | 3.00 | 0.60 | lubricant | 0.25-2 |
| Total Tablet Weight | 500.00 | 100.00 | | |

*45.1 mg of Pregabalin is based on purity equivalent to 45 mg of bioactive agent.

### Example 5

| Ingredients | mg/tab | % | Function | Range % |
|---|---|---|---|---|
| Amoxicillin | 344.4* | 34.44 | bioactive agent | 2-40% |
| Trihydrate | | | | |
| Microcrystalline | 270.6 | 27.06 | binder/filler | 1-80% |
| cellulose | | | | |
| Hypromellose | 125.00 | 12.5 | control release | 5-60% |
| 2208 | | | polymer | |
| Sodium Starch | 100.00 | 10.0 | disintegrant | 10-50% |
| Glycolate | | | | |
| polymethacrylate | 150.00 | 15.00 | enteric filler pH 5.5 | 1-75% |
| L100-55 | | | | |
| magnesium | 10.00 | 1.00 | lubricant | 0.25-2% |
| stearate | | | | |
| Total Tablet | 1000.00 | 100.00 | | |
| Weight | | | | |

*344.4 mg of Amoxicillin Trihydrate is 300 mg of free base equivalent.

### Example 6

| Ingredients | mg/tab | % | Function | Range % |
|---|---|---|---|---|
| Tramadol HCl | 113.9* | 15.19 | bioactive agent | 2-40 |
| Microcrystalline | 61.1 | 8.14 | binder/filler | 1-80 |
| cellulose | | | | |
| Hypromellose 2208 | 300.00 | 40 | control release | 5-60 |
| | | | polymer | |
| Sodium | 75.00 | 10.0 | disintegrant | 10-50 |
| Croscarmellose | | | | |
| polymethacrylate | 192.50 | 25.67 | enteric filler pH 5.5 | 1-75 |
| L100-55 | | | | |
| magnesium | 7.50 | 1.00 | lubricant | 0.25-2 |
| stearate | | | | |
| Total Tablet | 750.00 | 100.00 | | |
| Weight | | | | |

*113.9 mg of Tramadol HCl is 100 mg of free base equivalent.

### Example 7

| Ingredients | mg/tab | % | Function | Range % |
|---|---|---|---|---|
| Tramadol HCl | 113.9* | 15.19 | bioactive agent | 2-40 |
| 8 parts polyvinyl acetate & 2 | 159.85 | 21.31 | binder/filler/rigidity enhancer | 1-80 |
| parts polyvinylpyrrolidone or | | | | |
| Kollidon SR | | | | |
| Crospovidone | 187.5 | 25.00 | disintegrant | 10-50 |
| Polyethylene oxide WSR N- | 225 | 30 | control release polymer | 5-60 |
| 60K NF | | | | |
| Carbomer 71G or 971P | 56.25 | 7.5 | gelling agent/mucoadhesive | 0.5-20 |
| magnesium stearate NF | 7.50 | 1.0 | lubricant | 0.25-2 |
| Total Tablet Weight | 750.00 | 100.00 | | |

*113.9 mg of Tramadol HCl is 100 mg of free base bioactive agent.

### In-Vitro Dissolution Study

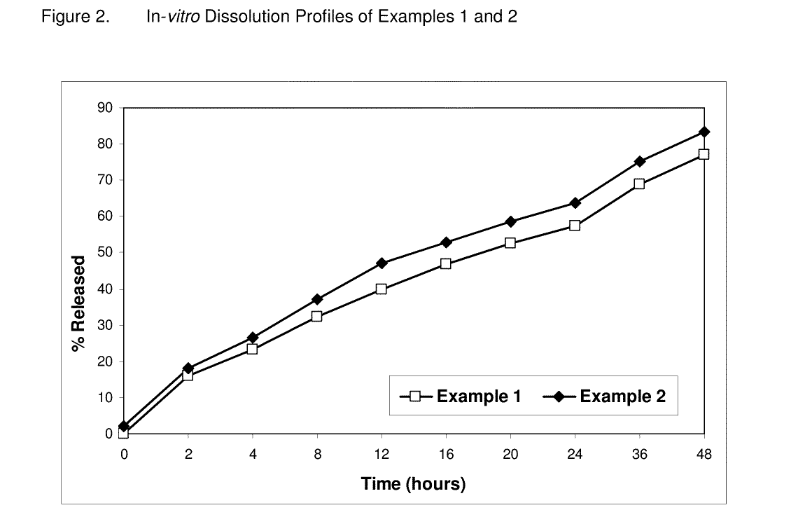

The in vitro dissolution release profiles for tablets containing bioactive agents (examples 1-7) are shown in FIG. 2-5. The dissolution method was performed using a USP I dissolution apparatus (Hanson SR8 plus) coupled with an auto sampler. The dissolution medium consisted of 900 mL citrate buffer (pH 3.6) or water maintained at 37±0.5° C. for 48 hours at 200 rpm. A 1.4 mL sample volume was withdrawn at 0, 2, 4, 8, 12, 16, 20, and 24 hours with some samples taken out to 36 and 48 hours. The hydrated tablet system dissolves drug and diffuses through the hydrogel matrix. The sustained and controlled release of bioactive agents was observed across the time profile. The bioactive agents were analyzed by UV-HPLC at a wavelength of 288 nm.

FIG. 2 illustrates in vitro dissolution profiles of Examples 1 and 2 of the present invention. In FIG. 2, the line with empty squares represents the in vitro dissolution profile of Example 1. The line with filled diamonds represents the in vitro dissolution profile of Example 2. A conventional immediate release tablet or capsule for Compound A in citrate buffer (pH 3.6) would have a complete drug release within 15 minutes. By this invention it is possible to extend the release from 15 minutes to about 48 hours (in-vitro).

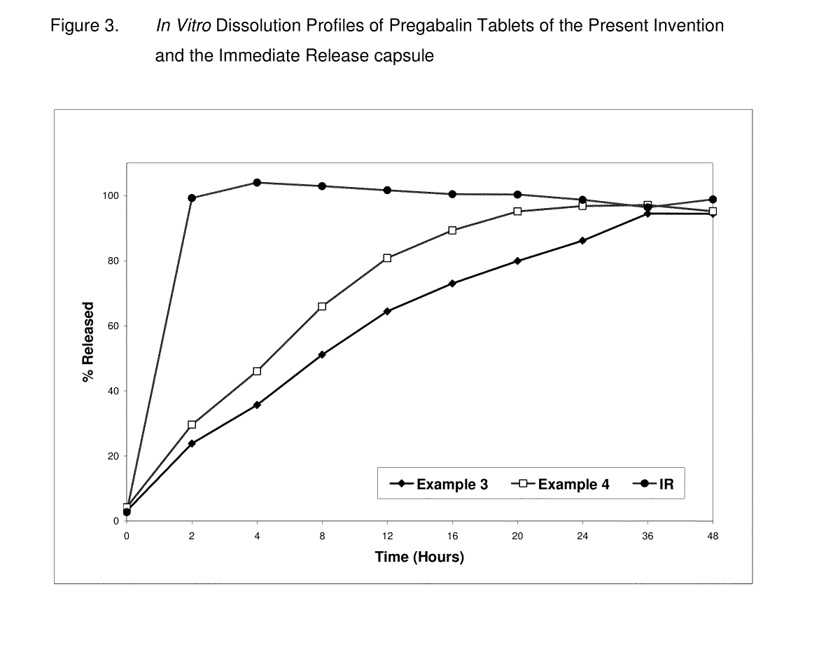

FIG. 3 illustrates in vitro dissolution profiles of pregabalin 45 mg tablets. In FIG. 3, the line with filled diamonds represents the in vitro dissolution profile of Example 3 of the present invention. The line with empty squares represents the in vitro dissolution profile of Example 4 of the present invention. The line with filled circles represents the in vitro dissolution profile of an immediate release pregabalin capsule. Pregabalin is currently used in pain management in Humans. Pregabalin under the trade name Lyrica® is administered in 2 or 3 doses per day. Pregabalin is commercially available, but the appropriate dose regimen for oral pregabalin in dogs is still unknown. Applying the technology of the present invention, it is possible to reducing the dosing frequency of pregabalin to once a day in canine as an anti-seizure option for dogs with epilepsy or as a pain reliever. One of the objects of the present invention is to use the veterinary composition of the present invention containing pregabalin for manufacturing of a medicament for the treatment of seizure, epilepsy or pains in animals including dogs. Another object of the present invention is to provide a method for the treatment of seizure, epilepsy or pains in animals including dogs by administering to the animals in need an effective amount of the veterinary composition of the present invention containing pregabalin.

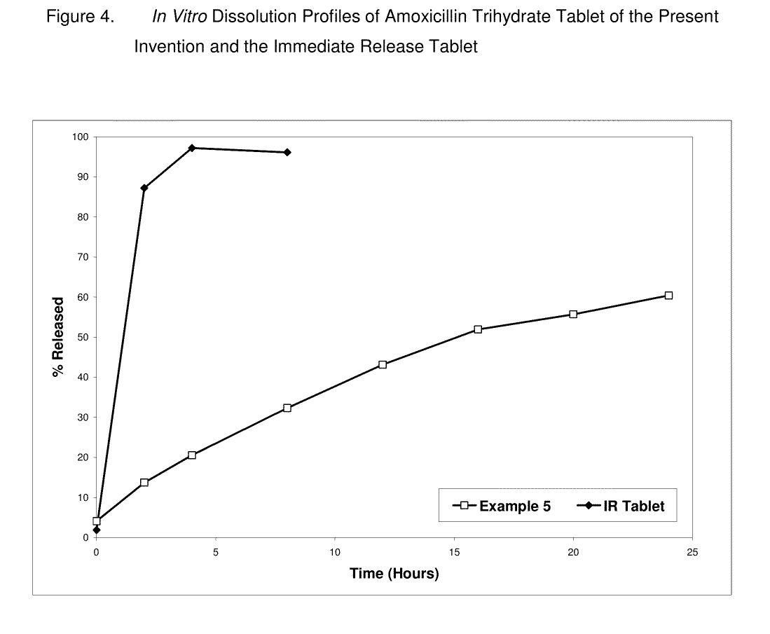

FIG. 4 illustrates in vitro dissolution profiles of amoxicillin trihydrate 300 mg tablets. In FIG. 4, the line with empty squares represents the in vitro dissolution profile of Example 5 of the present invention. The line with filled diamonds represents the in vitro dissolution profiles of an immediate release amoxicillin tablet. Amoxicillin is indicated for treatment in dogs for skin and soft-tissue infections such as wounds, abscesses, cellulitis, and superficial (juvenile) and deep pyoderma. It is also indicated for periodontal infections due to susceptible strains of both aerobic and anaerobic bacteria. At present the commercial products for canine treatment requires twice a day dosing. Though a controlled release Amoxicillin (Augmentin-XR) drug product is available for human use, it still calls for twice a day dosing. Applying the technology of the present invention, it is possible to reduce the dosing frequency of amoxicillin to once a day in dogs. One of the objects of the present invention is to use the veterinary composition of the present invention containing amoxicillin for manufacturing of a medicament for the treatment of skin and soft-tissue infections such as wounds, abscesses, cellulitis, superficial (juvenile) or deep pyoderma, and periodontal infections in animals including dogs. Another object of the present invention is to provide a method for the treatment of skin and soft-tissue infections such as wounds, abscesses, cellulitis, superficial (juvenile) or deep pyoderma, and periodontal infections in animals including dogs by administering to the animals in need an effective amount of the veterinary composition of the present invention containing amoxicillin.

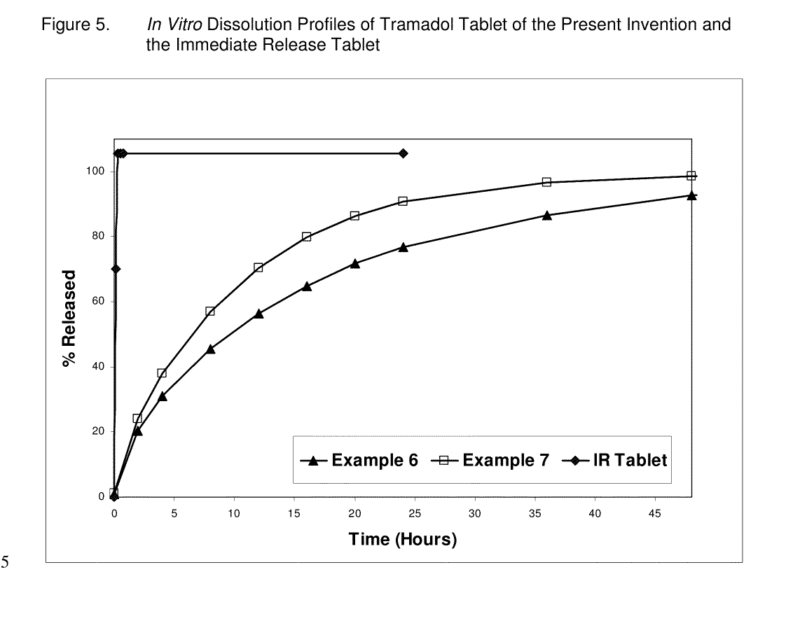

FIG. 5 illustrates In Vitro dissolution profiles of tramadol hydrochloride (HCl) 100 mg tablets. In FIG. 5, the line with filled triangles represents the in vitro dissolution profile of Example 6 of the present invention. The line with empty squares represents the in vitro dissolution profile of Example 7 of the present invention. The line with filled diamonds represents the in vitro dissolution profile of Tramadol 50 mg immediate release Tablet under the trademark Ultram®. Tramadol is a pain relieve that has been used by humans but has been introduced to the veterinary community to treat various pains including chronic pain and post-surgery pain in dogs and cats. Symptoms of canine arthritis can be controlled and treated using Tramadol for dogs. Tramadol is usually prescribed as immediate release tablets and administered as needed every four to six hours. Applying the technology of the present invention, it is possible to reducing the dosing frequency to once a day in dogs. One of the objects of the present invention is to use the veterinary composition of the present invention containing tramadol for manufacturing of a medicament for the treatment of various pains including chronic pain and post-surgery pain in animals including dogs. Another object of the present invention is to provide a method for the treatment of various pains including chronic pain and post-surgery pain in dogs in animals including dogs by administering to the animals in need an effective amount of the veterinary composition of the present invention containing tramadol.

### Pharmacokinetic Studies

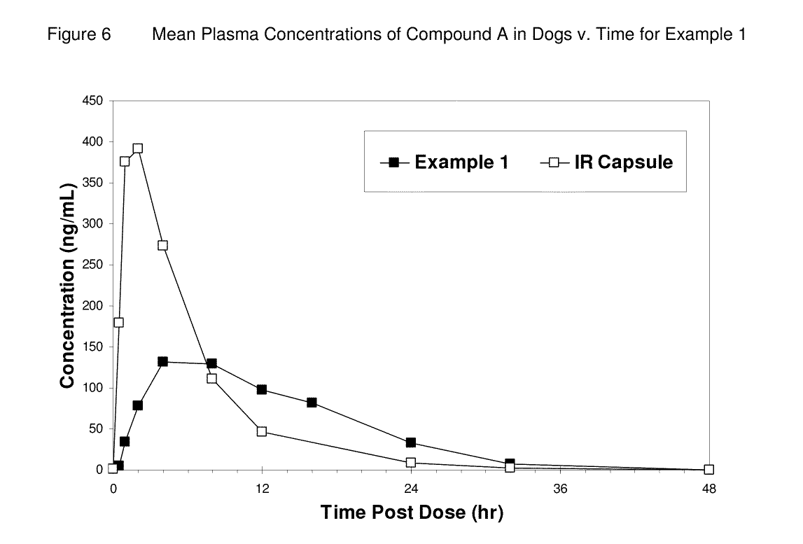

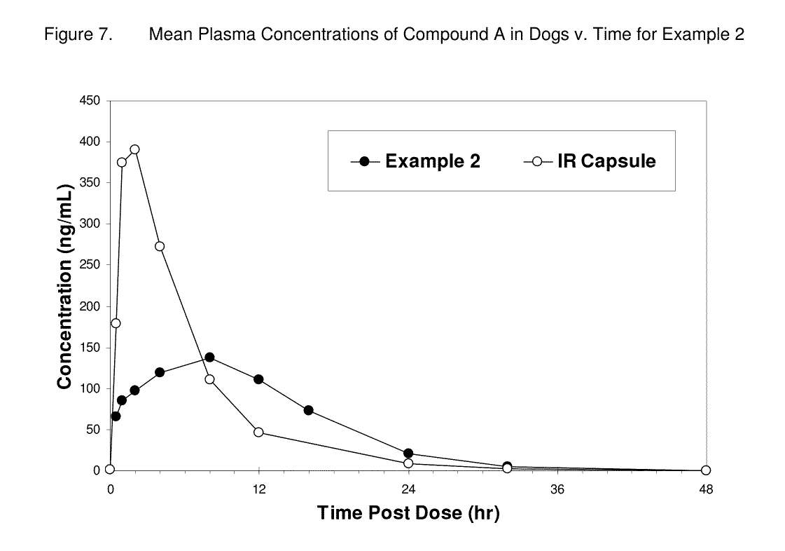

The composition of the present invention is capable of prolonging gastric retention time up to 16 hours in canines for once-daily oral administration. In a study for Compound-A, a parallel design pharmacokinetic study was carried out in canines in which the compositions of the present invention were compared to an immediate release formulation. Each treatment group consisted of five female beagles that were fed before administration of single oral 10.75 mg Compound-A maleate salt (equivalent of 8 mg of free base) dose in the form of either immediate release formulation or the tablets of current invention. Blood samples were collected at specified times for 72 hr following drug administration. At all sample collections, plasma concentrations of compound-A were determined, from which pharmacokinetics were evaluated and the data is presented in FIGS. 6 and 7. In FIG. 6, the line with empty squares represents the plasma drug concentration-time profiles for the immediate release capsule. The line with filled squares represents the plasma drug concentration-time profiles for Compound-A of Example 1 of this invention. As can be seen from FIG. 6, the Example 1 of this invention has an extended Tmax (4.8 h) when compared to immediate release dosage form (1.4 h). Similarly the mean residence time (MRT) of the Example 1 of this invention is longer (12 h) when compared to that of immediate release dosage form (4.8 h). And the Cmax of the Example 1 is several folds lower than the immediate release dosage form, which would provide a greater safety margin while the longer MRT would provide longer duration of efficacy. In FIG. 7, the line with empty circles represents the plasma drug concentration-time profiles for the immediate release capsule. The line with filled circles represents the plasma drug concentration-time profiles for Compound-A of Example 2 of this invention. As can be seen from FIG. 7, the Example 2 of this invention has an extended Tmax (5.2 h) when compared to immediate release dosage form (1.2 h). Similarly the mean residence time (MRT) of the Example 2 of this invention is longer (11.1 h) when compared to that of immediate release dosage form (4.8 h). And the Cmax of the Example 2 is several folds lower than the immediate release dosage form, which would provide a greater safety margin while the longer MRT would provide longer duration of efficacy.

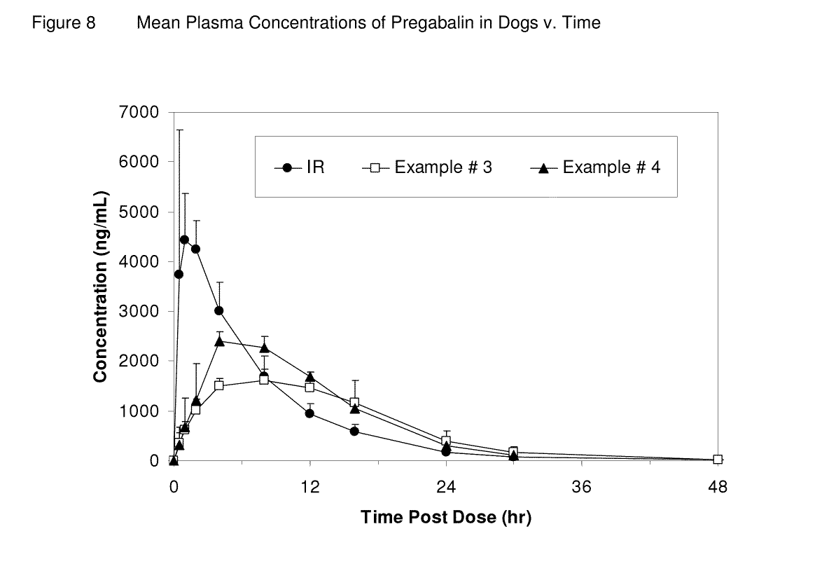

In another PK study for pregabalin, a parallel design pharmacokinetic study was carried out in canines in which the compositions of the present invention were compared to an immediate release formulation. Each treatment group consisted of five male beagles that were fed before administration of single oral 45 mg Pregabalin dose in the form of either immediate release formulation or the tablets of current invention. Blood samples were collected at specified times for 72 hr following drug administration. At all sample collections, plasma concentrations of Pregabalin were determined, from which pharmacokinetics were evaluated and the data is presented in FIG. 8.

In FIG. 8, the line with filled circles represents the plasma drug concentration-time profiles for the immediate release capsule. The line with open squares represents the plasma drug concentration-time profiles for Pregabalin of Example 3 of this invention. As can be seen from FIG. 8, the Example 3 of this invention has an extended Tmax (8.0 h) when compared to immediate release dosage form (1.3 h). Similarly the mean residence time (MRT) of the Example 3 of this invention is longer (12.4 h) when compared to that of immediate release dosage form (7.27 h). And the Cmax of the Example 3 is significantly lower than the immediate release dosage form, which would provide a greater safety margin while the longer MRT would provide longer duration of efficacy. The line with filled triangles represents the plasma drug concentration-time profiles for Pregabalin of Example 4 of this invention. As can be seen from FIG. 8, the Example 4 of this invention has an extended Tmax (4.0 h) when compared to immediate release dosage form (1.3 h). Similarly the mean residence time (MRT) of the Example 4 of this invention is longer (10.8 h) when compared to that of immediate release dosage form (7.27 h). And the Cmax of the Example 4 is considerably lower than the immediate release dosage form, which would provide a greater safety margin while the longer MRT would provide longer duration of efficacy.

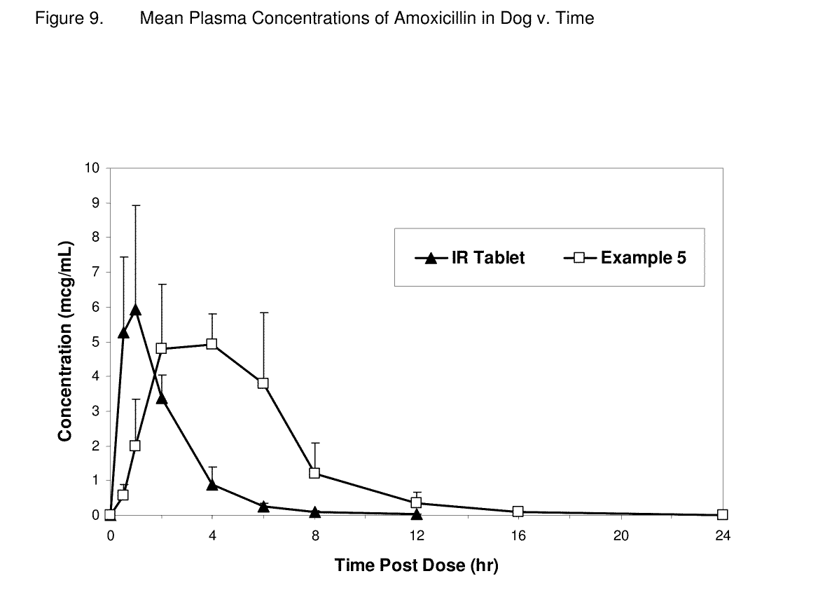

In another PK study for amoxicillin, a parallel design pharmacokinetic study was carried out in canines in which the compositions of the present invention were compared to an immediate release formation under the trade name Clavamox®. Each treatment group consisted of five male beagles that were fed before administration of either an oral 125 mg and 62.5 mg dose of Clavamox® as an immediate release tablet formulation or a single oral 300 mg Amoxicillin dose using the tablets of current invention. Blood samples were collected at specified times for 72 hr following drug administration. At all sample collections, plasma concentrations of Amoxicillin were determined, from which pharmacokinetics were evaluated and the data is presented in FIG. 9.

In FIG. 9, the line with the filled triangles represents the plasma drug concentration-time profiles for the immediate release tablets. The line with empty squares represents the plasma drug concentration-time profiles for Amoxicillin of Example 5 of this invention. As can be seen from FIG. 9, the Example 5 of this invention has an extended Tmax (3.5 h) when compared to immediate release dosage form (0.75 h). Similarly the mean residence time (MRT) of the Example 5 of this invention is longer (4.8 h) when compared to that of immediate release dosage form (2.03 h). And the Cmax of the Example 5 is lower than the immediate release dosage form, which would provide similar exposure while the longer MRT would provide longer duration of efficacy.

We claim:

1. A controlled-release veterinary composition in a form of an orally deliverable tablet prepared by direct compression comprising:
(a) at least one bioactive agent for veterinary use;
(b) a controlled release polymer selected from Hypromellose 2208 or Polyox WSR n-60; in an amount of about 5% to about 60% of the total weight of the tablet; and
(c) at least one disintegrant agent in an amount between about 10% to about 25% of the total weight of the tablet.

2. (canceled)

3. The composition of claim 1 wherein the bioactive agent is in an amount of about 2% to about 25% of the total weight of the tablet.

4. The composition of claim 1 which may further comprise one or more enteric fillers or enteric polymers with a pH range of about 5.5-9.0.

5. The bioactive agent of claim 1 selected from amoxicillin trihydrate, pregabalin, and N-methyl-1-{trans-4-[methyl(7H-pyrrolo[2,3-d]pyrimidin-4-yl)amino]cyclohexyl}methanesulfonamide.

6. The bioactive agent of claim 5 which is N-methyl-1-{trans-4-[methyl(7H-pyrrolo[2,3-d]pyrimidin-4-yl)amino]cyclohexyl}methanesulfonamide.

7. The composition of claim 1 wherein the polymer is Hypromellose 2208.

8. The composition of claim 1 wherein the polymer is Polyox WSR n-60.

9-11. (canceled)

12. The composition of claim 1 wherein the polymer is in an amount of from about 20% to about 40%, by weight of the tablet.

13. The composition of claim 1 wherein the disintegrant is selected from a group consisting of croscarmellose sodium, sodium carboxymethyl starch, cross linked povidone, and 2-hydroxypropyl ether (low substituted).

14. The composition of claim 1 wherein the disintegrant is in an amount from about 10% to about 40% of the total weight of the tablet.

15. (canceled)

16. The composition of claim 14 wherein the disintegrant is in an amount from about 10% to about 25% of the total weight of the tablet.

17. The composition of claim 1 further comprising veterinary acceptable excipients.

18. The composition of claim 1 which is capable of prolonging gastric retention time up to 16 hours in canines for once-daily oral administration.

19-20. (canceled)

21. Use of the veterinary composition of claim 6 for the treatment of allergic reactions, allergic dermatitis, atopic dermatitis, eczema, pruritus and inflammatory diseases in dogs.

22. (canceled)

23. Use of the veterinary composition of claim 5 for the treatment of skin and soft-tissue infections such as wounds, abscesses, cellulitis, superficial (juvenile) or deep pyoderma, and periodontal infections in dogs wherein the bioactive agent is amoxicillin trihydrate.

24. (canceled)
